(19) United States
(12) Patent Application Publication
(10) Pub. No.: US 2018/0042925 A1
(43) Pub. Date: Feb. 15, 2018

(54) METHODS FOR TREATING CANCER

(71) Applicant: CHILDREN'S HOSPITAL MEDICAL CENTER, Cincinnati, OH (US)

(72) Inventor: Biplab DASGUPTA, Cincinnati, OH (US)

(73) Assignee: Children's Hospital Medical Center, Cincinnati, OH (US)

(21) Appl. No.: 15/555,498

(22) PCT Filed: Mar. 4, 2016
(86) PCT No.: PCT/US16/20913

§ 371 Date: Sep. 2, 2017

Related U.S. Application Data
(60) Provisional application No. 62/128,256, filed on Mar. 4, 2015.

(51) Int. Cl. A61K 31/501; C12Q 1/68; A61K 45/06

(52) U.S. Cl. CPC A61K 31/501; A61K 45/06; C12Q 1/6886; C12Q 2600/156; C12Q 2600/106

(57) ABSTRACT

Some embodiments of the invention include methods to treat cancer in animals by administering an SCD inhibitor. Other embodiments include treating cancer in animals where the SCD gene inhibitor is absent in one or both chromatids by administering an SCD inhibitor. Still other embodiments include treating cancer in animals by administering an SCD inhibitor where the SCD gene is absent in one or both chromatids and the animal has a daily intake of one or more of (a) a specified amount of total fat, (b) a specified amount of total fatty acid, (c) a specified amount of total monounsaturated fatty acid, or (d) a specified amount of total oleic acid. Additional embodiments of the invention are also discussed herein.

### CROSS-REFERENCE TO RELATED APPLICATIONS

This application claims the benefit of U.S. Provisional Application No. 62/128,256, filed Mar. 4, 2015, entitled “METHODS OF TREATMENT OF GLIOBLASTOMA USING AN SCD1 (STEAROYL-COA DESATURASE) INHIBITOR” which is herein incorporated by reference in its entirety.

### BACKGROUND

Glioblastoma multiforme (GBM) is a malignant primary brain tumor in humans. It typically has poor patient outcome despite aggressive therapy including surgery, radiation, and chemotherapy. Other cancers (e.g., endometrial cancer, meanoma, prostate cancer, lung cancer, breast cancer, kidney cancer, or bladder cancer) can be non-responsive or lack sufficient response to certain therapies.

Stearoyl-CoA 9-Desaturase (SCD) is a protein found in many animals (e.g., mammals, primates, rodents, rats, mice, or humans). It can be used to produce the monounsaturated fatty acid oleic acid from the saturated fatty acid stearic acid. Oleic acid is typically one of the most abundant monounsaturated fatty acids and can be precursor for the formation of complex lipids. Lipids, such as oleic acid, can be used by cancer cells to, for example, sustain the relatively high cancer cell growth rate.

To date, treatment of cancers is limited. Accordingly, some embodiments of the present invention include treating cancer comprising administering an SCD inhibitor. Additional embodiments of the invention are also discussed herein.

### SUMMARY

Some embodiments of the present invention include methods for treating cancer in an animal comprising administering an SCD inhibitor to the animal, where an SCD gene is absent from one or both chromatids. In other embodiments, the SCD inhibitor can be an SCD1 inhibitor, a small molecule SCD1 inhibitor, or 3-[4-(2-chloro-5-fluorophenoxy)-1-piperidinyl]-6-(5-methyl-1,3,4-oxadiazol-2-yl)-pyridazine. In certain embodiments, the SCD gene can be an SCD1 gene. In yet other embodiments, the method can further comprise determining if the SCD gene is absent from one or both chromatids or the method can further comprise determining if the SCD gene is absent from one or both chromatids prior to administering the SCD inhibitor. The absence of the SCD gene from one or both chromatids can, in some instances, be determined by measuring the presence or absence of the PTEN gene in one or both chromatids or be determined by measuring the presence or absence of the SCD gene in one or both chromatids; measuring the presence or absence of the SCD gene, the presence or absence of the PTEN gene, or both can be performed using FISH, CISH, or a CNV array. In still other embodiments, the animal can have a daily intake of one or more of (a) total fat of no more than about 3 gm/kg body weight, (b) total fatty acid of no more than about 3 gm/kg body weight, (c) total monounsaturated fatty acid of no more than about 2 gm/kg body weight, or (d) total oleic acid of no more than about 2 gm/kg body fat; the animal can have a daily intake of one or more of (a) total fat of no more than about 1 gm/kg body weight, (b) total fatty acid of no more than about 1 gm/kg body weight, (c) total monounsaturated fatty acid of no more than about 1 gm/kg body weight, or (d) total oleic acid of no more than about 1 gm/kg body fat; the animal can have a daily intake of one or more of (a) total fat of no more than about 0.1 gm/kg body weight, (b) total fatty acid of no more than about 0.1 gm/kg body weight, (c) total monounsaturated fatty acid of no more than about 0.1 gm/kg body weight, or (d) total oleic acid of no more than about 0.1 gm/kg body fat; or the animal can have a daily intake of one or more of (a) total fat of no more than about 0.01 gm/kg body weight, (b) total fatty acid of no more than about 0.01 gm/kg body weight, (c) total monounsaturated fatty acid of no more than about 0.01 gm/kg body weight, or (d) total oleic acid of no more than about 0.01 gm/kg body fat. The intake can sometimes occur by ingestion, enteral feeding, parenteral feeding, or a combination thereof. In some embodiments, the animal can be human and the one or both chromatids can be part of chromosome 10. In other embodiments, the animal can be in need of the treatment. In still other embodiments, the method can be for treating glioblastoma multiforme, endometrial cancer, melanoma, prostate cancer, lung cancer, breast cancer, kidney cancer, or bladder cancer; the method can be for treating glioblastoma multiforme, endometrial cancer, or melanoma; the method can be for treating cancerous tumors, cancers that are inherited, or cancers resulting from an inherited predisposition; or the method can be for treating brain tumors. In certain instances, the animal can be a mammal, rodent, primate, rat, mouse, or human. In some embodiments, SCD inhibitor can be administered to the animal in an amount of from about 10 mg of SCD inhibitor/kg animal body weight to about 200 mg of SCD inhibitor/kg animal body weight. Still other embodiments can include administering using parenteral administration, a mucosal administration, intravenous administration, subcutaneous administration, topical administration, intradermal administration, oral administration, sublingual administration, intranasal administration, or intramuscular administration. In some instances, the treatment further can comprise one or more of surgical intervention, chemotherapy, radiation therapy, hormone therapies, immunotherapy, or adjuvant systematic therapies.

Other embodiments of the invention include methods for treating cancer in an animal comprising administering an SCD inhibitor to the animal; the animal can have a daily intake of one or more of (a) total fat of no more than about 3 gm/kg body weight, (b) total fatty acid of no more than about 3 gm/kg body weight, (c) total monounsaturated fatty acid of no more than about 2 gm/kg body weight, or (d) total oleic acid of no more than about 2 gm/kg body fat. In certain embodiments, the method can further comprise determining if the SCD gene is absent from one or both chromatids or the method can further comprise determining if the SCD gene is absent from one or both chromatids prior to administering the SCD inhibitor. The absence of the SCD gene from one or both chromatids can, in some instances, be determined by measuring the presence or absence of the PTEN gene in one or both chromatids or be determined by measuring the presence or absence of the SCD gene in one or both chromatids; measuring the presence or absence of the SCD gene, the presence or absence of the PTEN gene, or both can be performed using FISH, CISH, or a CNV array. In still other embodiments, the animal can have a daily intake of one or more of (a) total fat of no more than about 1 gm/kg body weight, (b) total fatty acid of no more than about 1 gm/kg body weight, (c) total monounsaturated fatty acid of no more than about 1 gm/kg body weight, or (d) total oleic acid of no more than about 1 gm/kg body fat; the animal can have a daily intake of one or more of (a) total fat of no more than about 0.1 gm/kg body weight, (b) total fatty acid of no more than about 0.1 gm/kg body weight, (c) total monounsaturated fatty acid of no more than about 0.1 gm/kg body weight, or (d) total oleic acid of no more than about 0.1 gm/kg body fat; or the animal can have a daily intake of one or more of (a) total fat of no more than about 0.01 gm/kg body weight, (b) total fatty acid of no more than about 0.01 gm/kg body weight, (c) total monounsaturated fatty acid of no more than about 0.01 gm/kg body weight, or (d) total oleic acid of no more than about 0.01 gm/kg body fat. The intake can sometimes occur by ingestion, enteral feeding, parenteral feeding, or a combination thereof. In some embodiments, the animal can be human and the one or both chromatids can be part of chromosome 10. In other embodiments, the animal can be in need of the treatment. In still other embodiments, the method can be for treating glioblastoma multiforme, endometrial cancer, melanoma, prostate cancer, lung cancer, breast cancer, kidney cancer, or bladder cancer; the method can be for treating glioblastoma multiforme, endometrial cancer, or melanoma; the method can be for treating cancerous tumors, cancers that are inherited, or cancers resulting from an inherited predisposition; or the method can be for treating brain tumors. In certain instances, the animal can be a mammal, rodent, primate, rat, mouse, or human. In some embodiments, SCD inhibitor can be administered to the animal in an amount of from about 10 mg of SCD inhibitor/kg animal body weight to about 200 mg of SCD inhibitor/kg animal body weight. Still other embodiments can include administering using parenteral administration, a mucosal administration, intravenous administration, subcutaneous administration, topical administration, intradermal administration, oral administration, sublingual administration, intranasal administration, or intramuscular administration. In some instances, the treatment can further comprise one or more of surgical intervention, chemotherapy, radiation therapy, hormone therapies, immunotherapy, or adjuvant systematic therapies. In certain embodiments, the SCD gene can be an SCD1 gene. In other embodiments, the SCD inhibitor can be an SCD1 inhibitor, a small molecule SCD1 inhibitor, or 3-[4-(2-chloro-5-fluorophenoxy)-1-piperidinyl]-6-(5-methyl-1,3,4-oxadiazol-2-yl)-pyridazine.

Still other embodiments of the invention include methods for treating cancer in an animal comprising determining whether the SCD gene is absent from one or both chromatids and administering an SCD inhibitor to the animal; the animal can have a daily intake of one or more of (a) total fat of no more than about 3 gm/kg body weight, (b) total fatty acid of no more than about 3 gm/kg body weight, (c) total monounsaturated fatty acid of no more than about 3 gm/kg body weight, or (d) total oleic acid of no more than about 2 gm/kg body fat. In certain aspects of the invention, the step of administering can be performed only if the SCD gene is absent from one or both chromatids, using the determining step. The absence of the SCD gene from one or both chromatids can, in some instances, be determined by measuring the presence or absence of the PTEN gene in one or both chromatids or be determined by measuring the presence or absence of the SCD gene in one or both chromatids; measuring the presence or absence of the SCD gene, the presence or absence of the PTEN gene, or both can be performed using FISH, CISH, or a CNV array. In some embodiments, the SCD gene can be absent from one or both chromatids. In still other embodiments, the animal can have a daily intake of one or more of (a) total fat of no more than about 1 gm/kg body weight, (b) total fatty acid of no more than about 1 gm/kg body weight, (c) total monounsaturated fatty acid of no more than about 1 gm/kg body weight, or (d) total oleic acid of no more than about 1 gm/kg body fat; the animal can have a daily intake of one or more of (a) total fat of no more than about 0.1 gm/kg body weight, (b) total fatty acid of no more than about 0.1 gm/kg body weight, (c) total monounsaturated fatty acid of no more than about 0.1 gm/kg body weight, or (d) total oleic acid of no more than about 0.1 gm/kg body fat; or the animal can have a daily intake of one or more of (a) total fat of no more than about 0.01 gm/kg body weight, (b) total fatty acid of no more than about 0.01 gm/kg body weight, (c) total monounsaturated fatty acid of no more than about 0.01 gm/kg body weight, or (d) total oleic acid of no more than about 0.01 gm/kg body fat. The intake can sometimes occur by ingestion, enteral feeding, parenteral feeding, or a combination thereof. In some embodiments, the animal can be human and the one or both chromatids can be part of chromosome 10. In other embodiments, the animal can be in need of the treatment. In still other embodiments, the method can be for treating glioblastoma multiforme, endometrial cancer, melanoma, prostate cancer, lung cancer, breast cancer, kidney cancer, or bladder cancer; the method can be for treating glioblastoma multiforme, endometrial cancer, or melanoma; the method can be for treating cancerous tumors, cancers that are inherited, or cancers resulting from an inherited predisposition; or the method can be for treating brain tumors. In certain embodiments, the SCD gene can be an SCD1 gene. In certain instances, the animal can be a mammal, rodent, primate, rat, mouse, or human. In other embodiments, the SCD inhibitor can be an SCD1 inhibitor, a small molecule SCD1 inhibitor, or 3-[4-(2-chloro-5-fluorophenoxy)-1-piperidinyl]-6-(5-methyl-1,3,4-oxadiazol-2-yl)-pyridazine. In some embodiments, SCD inhibitor can be administered to the animal in an amount of from about 10 mg of SCD inhibitor/kg animal body weight to about 200 mg of SCD inhibitor/kg animal body weight. Still other embodiments can include administering using parenteral administration, a mucosal administration, intravenous administration, subcutaneous administration, topical administration, intradermal administration, oral administration, sublingual administration, intranasal administration, or intramuscular administration. In some instances, the treatment can further comprise one or more of surgical intervention, chemotherapy, radiation therapy, hormone therapies, immunotherapy, or adjuvant systematic therapies.

Other embodiments of the invention are also discussed herein.

### BRIEF DESCRIPTION OF THE DRAWINGS

The following drawings form part of the present specification and are included to further demonstrate certain aspects of the present invention. The invention may be better understood by reference to one or more of these drawings in combination with the description of specific embodiments presented herein.

FIG. 1: SCD1 expression of Normal Human Astrocytes (NHA) and Diffuse Intrinsic Pontine Glioma cells (DIPG—a pediatric primary GBM cell line), and primary adult GBM cell lines AC17, GSC157, GSC83, GSC84, GSC326, G35, G62, G68, and G82.

FIG. 2: Inhibition of SCD1. (A) Genetic Inhibition of SCD1. Cell viability assays in the presence or absence of SCD1 shRNA show that genetic inhibition of SCD1 resulted in induction of cell death and growth inhibition. (B) Inhibition of SCD1 in G62 cells using CAY10566. Cell viability assays in the presence or absence of CAY10566 show that pharmacological inhibition of SCD1 resulted in induction of cell death and growth inhibition. (C) Inhibition of SCD1 in G68 cells using CAY10566. Cell viability assays in the presence or absence of CAY10566 show that pharmacological inhibition of SCD1 resulted in induction of cell death and growth inhibition.

FIG. 3: Oleate Rescue of SCD1-Inhibited GBM Cells. (A) BSA-conjugated oleic acid rescues SCD1-Inhibited G62 Cells. G62 cell viability assays in the presence or absence of CAY10566 and in the presence or absence of BSA-conjugated oleic acid (oleic acid is the product of the enzymatic reaction catalyzed by SCD1) show cell inhibition by CAY10566 treatment can be rescued by BSA-conjugated oleic acid. (B) BSA-conjugated oleic acid rescues SCD1-Inhibited G68 Cells. G68 cell viability assays in the presence or absence of CAY10566 and in the presence or absence of BSA-conjugated oleic acid show cell inhibition by CAY10566 treatment can be rescued by BSA-conjugated oleic acid.

FIG. 4: Sensitivity of high SCD1 GBM stem cell lines to SCD1 inhibitor. The different cell lines were exposed to an SCD1 inhibitor at varying concentrations. Percent cell viability was determined at 72 hours. The different cells lines are: black diamond, normal human astrocytes; gray cross, GSC157; black square, TS600; gray triangle, GBM39; open circle, TS1156; gray square, GBM157; black triangle, G62; gray circle, G68; and black cross, G82.

FIG. 5: Sensitivity of high SCD1 GBM stem cell lines to SCD1 inhibitor compared to endometrial cancer cell lines. (A) Different cell lines were exposed to an SCD1 inhibitor at varying concentrations. Percent cell viability was determined at 72 hours. The different cells lines are: black square, Ishikawa cell line; gray diamond, HEC50B; gray square, GBM157; black triangle, G62; gray circle, G68; and black cross, G82. (B) Images showing fluorescent in situ hybridization (FISH) of PTEN and SCD1 in an endometrial cancer cell line. The arrowheads point to PTEN or SCD1, as indicated. The arrows point to the chromosome 10 centromere. (C) Images showing fluorescent in situ hybridization (FISH) of PTEN and SCD1 in a glioblastoma cancer cell line. The arrowheads point to PTEN or SCD1, as indicated. The arrow points to the chromosome 10 centromere.

### DETAILED DESCRIPTION

Some embodiments of the invention include treating cancer in an animal comprising administering an SCD inhibitor.

SCD Inhibitors

As used herein, the term “SCD inhibitor” means any molecule that directly or indirectly (e.g., via allosteric binding) reduces a biological activity of SCD (e.g., SCD1). The biological activity can be any biological activity of SCD including but not limited to producing monounsaturated fatty acids from saturated fatty acids, producing oleic acid from stearic acid, producing palmitoleic acid from palimitic acid, or combinations thereof. The SCD inhibitor (e.g., SCD1 inhibitor) can act by any suitable mechanism including but not limited to binding to the SCD or fully or partially blocking binding of another molecule. The reduction in biological activity of SCD (e.g., SCD1) can be any detectable decrease including, for example, a decrease in activity of about 10%, about 20%, about 30%, about 40%, about 50%, about 60%, about 70%, about 75%, about 80%, about 85%, about 90%, about 95%, about 99%, or greater. Any suitable method can be used to measure biological activity of SCD (e.g., SCD1) including but not limited to those disclosed herein.

Any suitable SCD inhibitor (e.g., in humans, an SCD1 inhibitor or an SCD5 inhibitor) can be used in the methods disclosed herein. In some embodiments, the SCD inhibitor can be a small molecule SCD inhibitor (e.g., a small molecule SCD1 inhibitor) or an antibody SCD inhibitor (e.g., an antibody SCD1 inhibitor).

The term “small molecule SCD inhibitor” refers to any SCD inhibitor (e.g., SCD1 inhibitor) with a molecular mass of about 4000 daltons or less; in some embodiments, it is no more than about 2000 daltons, no more than about 1000 daltons, or no more than about 500 daltons. An example of a small molecule SCD inhibitor is CAY10566, which is 3-[4-(2-chloro-5-fluorophenoxy)-1-piperidinyl]-6-(5-methyl-1,3,4-oxadiazol-2-yl)-pyridazine and has a CAS Registry number of 944808-88-2. Other examples of small molecule SCD inhibitors, include but are not limited to those disclosed in US Patent Application Publication No. 2005/0119254 (which is hereby incorporated by reference in its entirety), US Patent Application Publication No. 2013/0096181 (which is hereby incorporated by reference in its entirety), US Patent Application Publication No. 2015/0307463 (which is hereby incorporated by reference in its entirety), U.S. Pat. No. 7,582,633 (which is hereby incorporated by reference in its entirety), U.S. Pat. No. 7,652,013 (which is hereby incorporated by reference in its entirety), U.S. Pat. No. 7,754,745 (which is hereby incorporated by reference in its entirety), U.S. Pat. No. 7,799,787 (which is hereby incorporated by reference in its entirety), U.S. Pat. No. 8,003,677 (which is hereby incorporated by reference in its entirety), U.S. Pat. No. 8,063,224 (which is hereby incorporated by reference in its entirety), U.S. Pat. No. 8,258,160 (which is hereby incorporated by reference in its entirety), U.S. Pat. No. 8,314,138 (which is hereby incorporated by reference in its entirety), U.S. Pat. No. 8,383,643 (which is hereby incorporated by reference in its entirety), U.S. Pat. No. 9,102,669 (which is hereby incorporated by reference in its entirety), U.S. Pat. No. 9,168,248 (which is hereby incorporated by reference in its entirety), U.S. Pat. No. 9,233,102 (which is hereby incorporated by reference in its entirety), and U.S. Pat. No. 9,238,658 (which is hereby incorporated by reference in its entirety).

The term “antibody” is used herein in the broadest sense and encompasses various antibody structures, including but not limited to monoclonal antibodies, polyclonal antibodies, multi specific antibodies (e.g., bispecific antibodies), human antibodies, humanized antibodies, chimeric antibodies, and antibody fragments so long as they exhibit the desired antigen-binding activity.

The term “antibody SCD inhibitor” refers to an antibody that is capable of binding SCD with sufficient affinity such that the antibody is useful as an inhibitor of SCD activity or as a therapeutic agent in targeting SCD. In one embodiment, the extent of binding of an antibody SCD inhibitor to an unrelated, non-SCD1 protein is less than about 10% of the binding of the antibody to SCD. In certain embodiments, an antibody SCD inhibitor binds to an epitope of SCD that is conserved among SCD from different species.

Compositions including Pharmaceutical Compositions

One or more SCD inhibitors (e.g., SCD1 inhibitors) can be part of a composition and can be in an amount (by weight of the total composition) of at least about 0.0001%, at least about 0.001%, at least about 0.10%, at least about 0.15%, at least about 0.20%, at least about 0.25%, at least about 0.50%, at least about 0.75%, at least about 1%, at least about 10%, at least about 25%, at least about 50%, at least about 75%, at least about 90%, at least about 95%, at least about 99%, at least about 99.99%, no more than about 75%, no more than about 90%, no more than about 95%, no more than about 99%, or no more than about 99.99%, from about 0.0001% to about 99%, from about 0.0001% to about 50%, from about 0.01% to about 95%, from about 1% to about 95%, from about 10% to about 90%, or from about 25% to about 75%.

One or more SCD inhibitors (e.g., SCD1 inhibitors) can be purified or isolated in an amount (by weight of the total composition) of at least about 0.0001%, at least about 0.001%, at least about 0.10%, at least about 0.15%, at least about 0.20%, at least about 0.25%, at least about 0.50%, at least about 0.75%, at least about 1%, at least about 10%, at least about 25%, at least about 50%, at least about 75%, at least about 90%, at least about 95%, at least about 99%, at least about 99.99%, no more than about 75%, no more than about 90%, no more than about 95%, no more than about 99%, no more than about 99.99%, from about 0.0001% to about 99%, from about 0.0001% to about 50%, from about 0.01% to about 95%, from about 1% to about 95%, from about 10% to about 90%, or from about 25% to about 75%.

Some embodiments of the present invention include compositions comprising one or more SCD inhibitors (e.g., SCD1 inhibitors). In certain embodiments, the composition is a pharmaceutical composition, such as compositions that are suitable for administration to animals (e.g., mammals, rodents, primates, monkeys, humans, canine, feline, porcine, mice, rabbits, or rats). In some instances, the pharmaceutical composition is non-toxic, does not cause side effects, or both. In some embodiments, there may be inherent side effects (e.g., it may harm the patient or may be toxic or harmful to some degree in some patients).

In some embodiments, one or more SCD inhibitors (e.g., SCD1 inhibitors) can be part of a pharmaceutical composition and can be in an amount of at least about 0.0001%, at least about 0.001%, at least about 0.10%, at least about 0.15%, at least about 0.20%, at least about 0.25%, at least about 0.50%, at least about 0.75%, at least about 1%, at least about 10%, at least about 25%, at least about 50%, at least about 75%, at least about 90%, at least about 95%, at least about 99%, at least about 99.99%, no more than about 75%, no more than about 90%, no more than about 95%, no more than about 99%, no more than about 99.99%, from about 0.001% to about 99%, from about 0.001% to about 50%, from about 0.1% to about 99%, from about 1% to about 95%, from about 10% to about 90%, or from about 25% to about 75%. In some embodiments, the pharmaceutical composition can be presented in a dosage form which is suitable for the topical, subcutaneous, intrathecal, intraperitoneal, oral, parenteral, rectal, cutaneous, nasal, vaginal, or ocular administration route. In other embodiments, the pharmaceutical composition can be presented in a dosage form which is suitable for parenteral administration, a mucosal administration, intravenous administration, subcutaneous administration, topical administration, intradermal administration, oral administration, sublingual administration, intranasal administration, or intramuscular administration. The pharmaceutical composition can be in the form of, for example, tablets, capsules, pills, powders granulates, suspensions, emulsions, solutions, gels (including hydrogels), pastes, ointments, creams, plasters, drenches, delivery devices, suppositories, enemas, injectables, implants (e.g., wafers comprising one or more SCD1 inhibitors), sprays, aerosols or other suitable forms.

In some embodiments, the pharmaceutical composition can include one or more formulary ingredients. A “formulary ingredient” can be any suitable ingredient (e.g., suitable for the drug(s), for the dosage of the drug(s), for the timing of release of the drugs(s), for the disease (e.g., cancer), for the disease state (e.g., stage of cancer), or for the delivery route) including, but not limited to, water (e.g., boiled water, distilled water, filtered water, pyrogen-free water, or water with chloroform), sugar (e.g., sucrose, glucose, mannitol, sorbitol, xylitol, or syrups made therefrom), ethanol, glycerol, glycols (e.g., propylene glycol), acetone, ethers, DMSO, surfactants (e.g., anionic surfactants, cationic surfactants, zwitterionic surfactants, or nonionic surfactants (e.g., polysorbates)), oils (e.g., animal oils, plant oils (e.g., coconut oil or arachis oil), or mineral oils), oil derivatives (e.g., ethyl oleate , glyceryl monostearate, or hydrogenated glycerides), excipients, preservatives (e.g., cysteine, methionine, antioxidants (e.g., vitamins (e.g., A, E, or C), selenium, retinyl palmitate, sodium citrate, citric acid, chloroform, or parabens, (e.g., methyl paraben or propyl paraben)), or combinations thereof.

In certain embodiments, pharmaceutical compositions can be formulated to release the active ingredient (e.g., one or more SCD inhibitors or one or more SCD1 inhibitors) substantially immediately upon the administration or any substantially predetermined time or time after administration. Such formulations can include, for example, controlled release formulations such as various controlled release compositions and coatings.

Other formulations (e.g., formulations of a pharmaceutical composition) can, in certain embodiments, include those incorporating the drug (or control release formulation) into food, food stuffs, feed, or drink.

Other embodiments of the invention can include methods of administering or treating an organism, which can involve treatment with an amount of at least one SCD inhibitor (e.g., SCD1 inhibitor) that is effective to treat the cancer, related condition, or related disorder that the organism has, or is suspected of having, or is susceptible to, or to bring about a desired physiological effect. In some embodiments, the composition or pharmaceutical composition comprises at least one SCD inhibitor (e.g., SCD1 inhibitor or an antibody SCD inhibitor) which can be administered to an animal (e.g., mammals, primates, monkeys, or humans) in an amount of about 0.01 to about 15 mg/kg body weight, about 0.1 to about 10 mg/kg body weight, about 0.5 to about 7 mg/kg body weight, about 0.01 mg/kg, about 0.05 mg/kg, about 0.1 mg/kg, about 0.5 mg/kg, about 1 mg/kg, about 3 mg/kg, about 5 mg/kg, about 5.5 mg/kg, about 6 mg/kg, about 6.5 mg/kg, about 7 mg/kg, about 7.5 mg/kg, about 8 mg/kg, about 10 mg/kg, about 12 mg/kg, or about 15 mg/kg. In regard to some conditions, the dosage can be about 0.5 mg/kg human body weight or about 6.5 mg/kg human body weight. In some instances, some animals (e.g., mammals, rodents, mice, rabbits, feline, porcine, or canine) can be administered a dosage of about 0.01 to about 15 mg/kg body weight, about 0.1 to about 10 mg/kg body weight, about 0.5 to about 7 mg/kg body weight, about 0.01 mg/kg, about 0.05 mg/kg, about 0.1 mg/kg, about 1 mg/kg, about 5 mg/kg, about 10 mg/kg, about 20 mg/kg, about 30 mg/kg, about 40 mg/kg, about 50 mg/kg, about 80 mg/kg, about 100 mg/kg, or about 150 mg/kg. In some embodiments, the composition or pharmaceutical composition comprises at least one SCD inhibitor (e.g., SCD1 inhibitor or small molecule SCD inhibitor) which can be administered to an animal (e.g., mammals, primates, monkeys, or humans) in an amount of about 1 to about 1000 mg/kg body weight, about 5 to about 500 mg/kg body weight, about 10 to about 200 mg/kg body weight, about 25 to about 100 mg/kg body weight, about 1 mg/kg, about 2 mg/kg, about 5 mg/kg, about 10 mg/kg, about 25 mg/kg, about 50 mg/kg, about 100 mg/kg, about 150 mg/kg, about 200 mg/kg, about 300 mg/kg, about 400 mg/kg, about 500 mg/kg, about 600 mg/kg, about 700 mg/kg, about 800 mg/kg, about 900 mg/kg, or about 1000 mg/kg. In regard to some conditions, the dosage can be about 20 mg/kg human body weight or about 100 mg/kg human body weight. In some instances, the composition or pharmaceutical composition comprises at least one SCD inhibitor (e.g., SCD1 inhibitor or small molecule SCD inhibitor) which can be administered to an animal (e.g., mammals, rodents, mice, rabbits, feline, porcine, or canine) in an amount of about 1 to about 1000 mg/kg body weight, about 5 to about 500 mg/kg body weight, about 10 to about 200 mg/kg body weight, about 25 to about 100 mg/kg body weight, about 1 mg/kg, about 2 mg/kg, about 5 mg/kg, about 10 mg/kg, about 25 mg/kg, about 50 mg/kg, about 100 mg/kg, about 150 mg/kg, about 200 mg/kg, about 300 mg/kg, about 400 mg/kg, about 500 mg/kg, about 600 mg/kg, about 700 mg/kg, about 800 mg/kg, about 900 mg/kg, or about 1000 mg/kg. Of course, those skilled in the art will appreciate that it is possible to employ many concentrations in the methods of the present invention, and using, in part, the guidance provided herein, will be able to adjust and test any number of concentrations in order to find one that achieves the desired result in a given circumstance. In other embodiments, the compounds of the invention can be administered in combination with one or more other therapeutic agents for a given cancer, related condition, or related disorder.

In some embodiments, the compositions can include a unit dose of one or more SCD inhibitors (e.g., SCD1 inhibitors) in combination with a pharmaceutically acceptable carrier and, in addition, can include other medicinal agents, pharmaceutical agents, carriers, adjuvants, diluents, and excipients. In certain embodiments, the carrier, vehicle or excipient can facilitate administration, delivery and/or improve preservation of the composition. In other embodiments, the one or more carriers, include but are not limited to, saline solutions such as normal saline, Ringer's solution, PBS (phosphate-buffered saline), and generally mixtures of various salts including potassium and phosphate salts with or without sugar additives such as glucose. Carriers can include aqueous and non-aqueous sterile injection solutions that can contain antioxidants, buffers, bacteriostats, bactericidal antibiotics, and solutes that render the formulation isotonic with the bodily fluids of the intended recipient; and aqueous and non-aqueous sterile suspensions, which can include suspending agents and thickening agents. In other embodiments, the one or more excipients can include, but are not limited to water, saline, dextrose, glycerol, ethanol, or the like, and combinations thereof. Nontoxic auxiliary substances, such as wetting agents, buffers, or emulsifiers may also be added to the composition. Oral formulations can include such normally employed excipients as, for example, pharmaceutical grades of mannitol, lactose, starch, magnesium stearate, sodium saccharine, cellulose, and magnesium carbonate.

Some administrations include injection (e.g., some forms of parenteral administration). Sterile injectables can be prepared in conventional forms, either as liquid solutions or suspensions, solid forms suitable for solution or suspension in liquid prior to injection, or as emulsions.

Administration Routes and Treatments of Disease

The SCD inhibitors (e.g., SCD1 inhibitors) of the invention can be administered to an animal by any number of suitable administration routes or formulations. The SCD inhibitors (e.g., SCD1 inhibitors) of the invention can also be used to treat an animal for a variety of diseases (e.g., cancer). Animals include but are not limited to mammals, rodents, primates, monkeys (e.g., macaque, rhesus macaque, or pig tail macaque), humans, canine, feline, bovine, porcine, avian (e.g., chicken), mice, rabbits, and rats. As used herein, the term “subject” refers to both human and animal subjects.

The route of administration of the SCD inhibitors (e.g., SCD1 inhibitors) of the invention can be of any suitable route. Administration routes can be, but are not limited to the oral route, the parenteral route, the cutaneous route, the nasal route, the rectal route, the vaginal route, and the ocular route. In other embodiments, administration routes can be parenteral administration, a mucosal administration, intravenous administration, subcutaneous administration, topical administration, intradermal administration, oral administration, sublingual administration, intranasal administration, or intramuscular administration. The choice of administration route can depend on the SCD inhibitor (e.g., SCD1 inhibitor) identity (e.g., the physical and chemical properties of the SCD inhibitor or SCD1 inhibitor) as well as the age and weight of the animal, the particular cancer, the severity of the cancer, and the stage of the cancer. Of course, combinations of administration routes can be administered, as desired.

Some embodiments of the invention include a method for providing a subject with a composition comprising an SCD inhibitor (e.g., SCD1 inhibitor) described herein (e.g., a pharmaceutical composition) which comprises one or more administrations of one or more such compositions; the compositions may be the same or different if there is more than one administration.

Cancers that can be treated in an animal (e.g., mammals, porcine, canine, avian (e.g., chicken), bovine, feline, primates, rodents, monkeys, rabbits, mice, rats, and humans) using the SCD inhibitors (e.g., SCD1 inhibitors) include, but are not limited to cancerous tumors (e.g., brain tumors), cancers that are inherited, cancers resulting from an inherited predisposition, cancers resulting from absence of one or more tumor suppressor genes, or cancers resulting from absence of one or more PTEN genes. Cancers that can be treated include, but are not limited to, glioblastoma multiforme, endometrial cancer, melanoma, prostate cancer, lung cancer, breast cancer, kidney cancer, bladder cancer, basal cell carcinoma, thyroid cancer, squamous cell carcinoma, neuroblastoma, ovarian cancer, renal cell carcinoma, hepatocellular carcinoma, colon cancer, pancreatic cancer, chronic lymphocytic leukemia (CLL), acute lymphoblastic leukemia, rhabdomyosarcoma, meningioma, gastric cancer, Glioma, oral cancer, nasopharyngeal carcinoma, rectal cancer, stomach cancer, uterine cancer, and leukemias. In some embodiments, cancers that can be treated include, but are not limited to, glioblastoma multiforme, endometrial cancer, melanoma, prostate cancer, lung cancer, breast cancer, kidney cancer, and bladder cancer. Animals that can be treated include but are not limited to mammals, rodents, primates, monkeys (e.g., macaque, rhesus macaque, pig tail macaque), humans, canine, feline, porcine, avian (e.g., chicken), bovine, mice, rabbits, and rats. As used herein, the term “subject” refers to both human and animal subjects. In some instances, the animal is in need of the treatment (e.g., by showing signs of disease or cancer, or by having a cancerous tumor).

As used herein, the term “treating” (and its variations, such as “treatment”) is to be considered in its broadest context. In particular, the term “treating” does not necessarily imply that an animal is treated until total recovery. Accordingly, “treating” includes amelioration of the symptoms, relief from the symptoms or effects associated with a condition, decrease in severity of a condition, or preventing, preventively ameliorating symptoms, or otherwise reducing the risk of developing a particular condition. As used herein, reference to “treating” an animal includes but is not limited to prophylactic treatment and therapeutic treatment. Any of the compositions (e.g., pharmaceutical compositions) described herein can be used to treat an animal.

As related to treating cancer (e.g., glioblastoma multiforme, endometrial cancer, melanoma, prostate cancer, lung cancer, breast cancer, kidney cancer, or bladder cancer), treating can include but is not limited to prophylactic treatment and therapeutic treatment. As such, treatment can include, but is not limited to: preventing cancer (e.g., glioblastoma multiforme, endometrial cancer, melanoma, prostate cancer, lung cancer, breast cancer, kidney cancer, or bladder cancer); reducing the risk of cancer (e.g., glioblastoma multiforme, endometrial cancer, melanoma, prostate cancer, lung cancer, breast cancer, kidney cancer, or bladder cancer); ameliorating or relieving symptoms of cancer (e.g., glioblastoma multiforme, endometrial cancer, melanoma, prostate cancer, lung cancer, breast cancer, kidney cancer, or bladder cancer); eliciting a bodily response against cancer (e.g., glioblastoma multiforme, endometrial cancer, melanoma, prostate cancer, lung cancer, breast cancer, kidney cancer, or bladder cancer); inhibiting the development or progression of cancer (e.g., glioblastoma multiforme, endometrial cancer, melanoma, prostate cancer, lung cancer, breast cancer, kidney cancer, or bladder cancer); inhibiting or preventing the onset of symptoms associated with cancer (e.g., glioblastoma multiforme, endometrial cancer, melanoma, prostate cancer, lung cancer, breast cancer, kidney cancer, or bladder cancer); reducing the severity of cancer (e.g., glioblastoma multiforme, endometrial cancer, melanoma, prostate cancer, lung cancer, breast cancer, kidney cancer, or bladder cancer); and causing a regression of cancer (e.g., glioblastoma multiforme, endometrial cancer, melanoma, prostate cancer, lung cancer, breast cancer, kidney cancer, or bladder cancer) or one or more of the symptoms associated with cancer (e.g., a decrease in tumor size). In some embodiments, treating does not include prophylactic treatment (e.g., preventing or ameliorating future cancer).

Treatment of an animal can occur using any suitable administration method (such as those disclosed herein) and using any suitable amount of SCD inhibitor or SCD1 inhibitor (such as those disclosed herein). In some embodiments, methods of treatment comprise treating an animal for cancer (e.g., glioblastoma multiforme, endometrial cancer, melanoma, prostate cancer, lung cancer, breast cancer, kidney cancer, or bladder cancer). Some embodiments of the invention include a method for treating a subject (e.g., an animal such as a human or primate) with a composition comprising an SCD inhibitor or SCD1 inhibitor described herein (e.g., a pharmaceutical composition) which comprises one or more administrations of one or more such compositions; the compositions may be the same or different if there is more than one administration.

In some embodiments, the method of treatment includes administering an effective amount of a composition comprising an SCD inhibitor (e.g., an SCD1 inhibitor). As used herein, the term “effective amount” refers to a dosage or a series of dosages sufficient to affect treatment (e.g., to treat cancer, such as but not limited to glioblastoma multiforme, endometrial cancer, melanoma, prostate cancer, lung cancer, breast cancer, kidney cancer, or bladder cancer) in an animal. In some embodiments, an effective amount can encompass a therapeutically effective amount, as disclosed herein. In certain embodiments, an effective amount can vary depending on the subject and the particular treatment being affected. The exact amount that is required can, for example, vary from subject to subject, depending on the age and general condition of the subject, the particular adjuvant being used (if applicable), administration protocol, and the like. As such, the effective amount can, for example, vary based on the particular circumstances, and an appropriate effective amount can be determined in a particular case. An effective amount can, for example, include any dosage or composition amount disclosed herein. In some embodiments, an effective amount of at least one SCD inhibitor, SCD1 inhibitor, or antibody SCD inhibitor (which can be administered to an animal such as mammals, primates, monkeys or humans) can be an amount of about 0.01 to about 15 mg/kg body weight, about 0.1 to about 10 mg/kg body weight, about 0.5 to about 7 mg/kg body weight, about 0.01 mg/kg, about 0.05 mg/kg, about 0.1 mg/kg, about 0.5 mg/kg, about 1 mg/kg, about 3 mg/kg, about 5 mg/kg, about 5.5 mg/kg, about 6 mg/kg, about 6.5 mg/kg, about 7 mg/kg, about 7.5 mg/kg, about 8 mg/kg, about 10 mg/kg, about 12 mg/kg, or about 15 mg/kg. In regard to some embodiments, the dosage can be about 0.5 mg/kg human body weight or about 6.5 mg/kg human body weight. In some instances, an effective amount of at least one SCD inhibitor, SCD1 inhibitor, or antibody SCD inhibitor (which can be administered to an animal such as mammals, rodents, mice, rabbits, feline, porcine, or canine) can be an amount of about 0.01 to about 15 mg/kg body weight, about 0.1 to about 10 mg/kg body weight, about 0.5 to about 7 mg/kg body weight, about 0.01 mg/kg, about 0.05 mg/kg, about 0.1 mg/kg, about 1 mg/kg, about 5 mg/kg, about 10 mg/kg, about 20 mg/kg, about 30 mg/kg, about 40 mg/kg, about 50 mg/kg, about 80 mg/kg, about 100 mg/kg, or about 150 mg/kg. In some embodiments, an effective amount of at least one SCD inhibitor, SCD1 inhibitor, or small molecule SCD inhibitor (which can be administered to an animal such as mammals, primates, monkeys or humans) can be an amount of about 1 to about 1000 mg/kg body weight, about 5 to about 500 mg/kg body weight, about 10 to about 200 mg/kg body weight, about 25 to about 100 mg/kg body weight, about 1 mg/kg, about 2 mg/kg, about 5 mg/kg, about 10 mg/kg, about 25 mg/kg, about 50 mg/kg, about 100 mg/kg, about 150 mg/kg, about 200 mg/kg, about 300 mg/kg, about 400 mg/kg, about 500 mg/kg, about 600 mg/kg, about 700 mg/kg, about 800 mg/kg, about 900 mg/kg, or about 1000 mg/kg. In regard to some conditions, the dosage can be about 20 mg/kg human body weight or about 100 mg/kg human body weight. In some instances, an effective amount of at least one SCD inhibitor, SCD1 inhibitor, or small molecule SCD inhibitor (which can be administered to an animal such as mammals, rodents, mice, rabbits, feline, porcine, or canine) can be an amount of about 1 to about 1000 mg/kg body weight, about 5 to about 500 mg/kg body weight, about 10 to about 200 mg/kg body weight, about 25 to about 100 mg/kg body weight, about 1 mg/kg, about 2 mg/kg, about 5 mg/kg, about 10 mg/kg, about 25 mg/kg, about 50 mg/kg, about 100 mg/kg, about 150 mg/kg, about 200 mg/kg, about 300 mg/kg, about 400 mg/kg, about 500 mg/kg, about 600 mg/kg, about 700 mg/kg, about 800 mg/kg, about 900 mg/kg, or about 1000 mg/kg.

“Therapeutically effective amount” means an amount effective to achieve a desired and/or beneficial effect (e.g., decreasing tumor size). A therapeutically effective amount can be administered in one or more administrations. For some purposes of this invention, a therapeutically effective amount is an amount appropriate to treat an indication (e.g., to treat cancer). By treating an indication is meant achieving any desirable effect, such as one or more of palliate, ameliorate, stabilize, reverse, slow, or delay disease (e.g., cancer) progression, increase the quality of life, or to prolong life. Such achievement can be measured by any method known in the art, such as but not limited to measurement of tumor size.

In some embodiments, the treatments can also include one or more of surgical intervention, chemotherapy, radiation therapy, hormone therapies, immunotherapy, and adjuvant systematic therapies. Adjuvants may include but are not limited to chemotherapy (e.g., temozolomide), radiation therapy, antiangiogenic therapy (e.g., bevacizumab), and hormone therapies, such as administration of LHRH agonists; antiestrogens, such as tamoxifen; high-dose progestogens; aromatase inhibitors; and/or adrenalectomy. Chemotherapy can be used as a single-agent or as a combination with known or new therapies.

In some embodiments, the administration of at least one SCD inhibitor (e.g., at least one SCD1 inhibitor) is an adjuvant cancer therapy or part of an adjuvant cancer therapy. Adjuvant treatments include treatments by the mechanisms disclosed herein and of cancers as disclosed herein, including, but not limited to tumors. Corresponding primary therapies can include, but are not limited to, surgery, chemotherapy, or radiation therapy. In some instances, the adjuvant treatment can be a combination of chemokine receptor antagonists with traditional chemotoxic agents or with immunotherapy that increases the specificity of treatment to the cancer and potentially limits additional systemic side effects. In still other embodiments, SCD inhibitors (e.g., SCD1 inhibitors) can be used as adjuvant with other chemotherapeutic agents. The use of an SCD inhibitor therapy (e.g., SCD1 inhibitor therapy) may, in some instances, reduce the duration of the dose of both drugs and drug combinations reducing the side effects. Limiting daily intake of one or more of total fat, total fatty acid, total monounsaturated fatty acid, or oleic acid can also be part of any of the adjuvant treatments.

Some embodiments of the invention include methods for treating cancer in an animal comprising administering an SCD inhibitor (e.g., SCD1 inhibitor) to the animal (e.g., a mammal, rodent, or primate), wherein an SCD gene (e.g., an SCD1 gene; in humans the SCD1 gene is NCBI Gene ID: 6319) is absent from one or both chromatids (e.g., chromosome 10 in humans). In certain embodiments, the animal is in need of the treatment thereof. In other embodiments, when the phosphatase and tensin homolog (PTEN) gene (in humans, the PTEN gene is NCBI Gene ID: 5728) is absent on the chromatid, the SCD gene (e.g., SCD1 gene) is also absent on that chromatid; this can, in some instances, result from the close proximity of the PTEN gene to the SCD gene (e.g., SCD1 gene) on the chromatid. The determination of whether an SCD gene (e.g., SCD1 gene) is absent from one or both chromatids can be performed using any suitable method including but not limited to (a) measuring the presence or absence of the PTEN gene in one or both chromatids (e.g., chromosome 10 in human), (b) measuring the presence or absence of the SCD gene (e.g., SCD1 gene) in one or both chromatids (e.g., chromosome 10 in human), or (c) both. In certain embodiments, measuring the presence or absence of the SCD gene (e.g., SCD1 gene), the PTEN gene, or both can be performed using any suitable method including but not limited to immunohistochemical (IHC) methods, Western blot analysis, immunoprecipitation, molecular binding assays, ELISA, ELIFA, fluorescence activated cell sorting (FACS), MassARRAY, proteomics, quantitative blood based assays (as for example Serum ELISA), biochemical enzymatic activity assays, in situ hybridization, Northern analysis, polymerase chain reaction (PCR) including quantitative real time PCR (qRT-PCR) and other amplification type detection methods, including, for example, branched DNA, SISBA, TMA), RNA-Seq, Fluorescence In Situ Hybridization (FISH), Chromogenic In Situ Hybridization (CISH), microarray analysis, gene expression profiling, serial analysis of gene expression (SAGE), a multiplexed immunoassay (e.g., those available from Rules Based Medicine or Mesa Scale Discovery (MSD)), a CNV array (e.g., an array to determine copy number variation) or combinations thereof, as well as any one of the wide variety of assays that can be performed by protein, gene, and/or tissue array analysis. In some embodiments, FISH, CISH, a multiplexed immunoassay, a CNV array, or combinations thereof can be used to measure the presence or absence of the SCD gene (e.g., the SCD1 gene), the PTEN gene, or both. In yet other embodiments, determining if an SCD gene (e.g., SCD1 gene) is absent from one or both chromatids (e.g., chromosome 10 in humans) may or may not be performed in the method (e.g., prior to administering one or more SCD inhibitors (e.g., one or more SCD1 inhibitors)). In still other embodiments, determining if an SCD gene (e.g., an SCD1 gene) is absent from one or both chromatids (e.g., chromosome 10 in humans) is performed prior to or after administering an SCD inhibitor (e.g., an SCD1 inhibitor).

In some embodiments, the method of treatment can include the animal having a daily intake of one or more of: (a) a specified amount of total fat (e.g., no more than about 3 gm/kg body weight of the animal), (b) a specified amount of total fatty acid (e.g., no more than about 3 gm/kg body weight of the animal), (c) a specified amount of total monounsaturated fatty acid (e.g., no more than about 2 gm/kg body weight of the animal), or (d) a specified amount of total oleic acid (e.g., no more than about 2 gm/kg body weight of the animal).

The term “total fat” as used herein is defined to include all lipids such as but not limited to triglycerides, fatty acids, sterols (e.g., cholesterol), sphingolipids, and phospholipids. Total fat encompasses total fatty acid. In some embodiments, the method of treatment includes a daily intake of total fat of no more than about 3 gm total fat/kg body weight of the animal. In other embodiments, the method of treatment includes a daily intake of total fat of about 0, about 0.001, about 0.01, about 0.02, about 0.03, about 0.04, about 0.05, about 0.06, about 0.07, about 0.08, about 0.09, about 0.1, about 0.2, about 0.3, about 0.4, about 0.5, about 0.6, about 0.7, about 0.8, about 0.9, about 1, about 1.1, about 1.2, about 1.3, about 1.4, about 1.5, about 1.6, about 1.7, about 1.8, about 1.9, about 2, about 2.1, about 2.2, about 2.3, about 2.4, about 2.5, about 2.6, about 2.7, about 2.8, about 2.9, about 3, from about 0 to about 3, from about 0 to about 2.5, from about 0 to about 2, from about 0 to about 1.5, from about 0 to about 1, from about 0 to about 0.5, from about 0 to about 0.3, from about 0 to about 0.2, from about 0 to about 0.1, from about 0 to about 0.05, from about 0 to about 0.01, from about 0 to about 0.001, no more than about 3, no more than about 2, no more than about 1.5, no more than about 1, no more than about 0.5, no more than about 0.3, no more than about 0.2, no more than about 0.1, no more than about 0.05, no more than about 0.01, or no more than about 0.001 gm total fat/kg body weight of the animal.

Any suitable method can be used to determine or measure total fat including but not limited to one or more of: extraction methods (e.g., solvent based extraction methods or nonsolvent liquid extraction methods such as the Babcock method or the Gerber method), hydrolysis methods, chemical techniques (e.g., Iodine value determination (e.g., using the Wijs method) or saponification number determination), mass spectrometry, gas chromatography, high pressure liquid chromatography, thin layer chromatography, nuclear magnetic resonance, or combinations thereof. In some embodiments, use of information provided by other sources such as but not limited to information found in books, in tables, using apps, using websites, or in packaging with a food item (such as nutritional labels including but not limited to those required by the US Food and Drug Administration, such as pursuant to 21 C.F.R. § 101 (e.g., § 101.9)) can be used as a method to determine total fat in a food item. One or more of these methods can be used to determine or measure total fat for one food item, for several food items, or for all the food items used to calculate the daily intake of total fat.

The term “total fatty acid” as used herein is defined to include all fatty acids, such as but not limited to saturated fatty acids, cis monounsaturated fatty acids, cis polyunsatuyrated fatty acids (e.g., n-6 fatty acid and n-3 fatty acids), trans fatty acids, and those fatty acids found in glycerides (e.g., triglycerides, diglycerides, and monoglycerides). Total fatty acid encompasses total monounsaturated fatty acid. In some embodiments, the method of treatment includes a daily intake of total fatty acid of no more than about 3 gm total fatty acid/kg body weight of the animal. In other embodiments, the method of treatment includes a daily intake of total fatty acid of about 0, about 0.001, about 0.01, about 0.02, about 0.03, about 0.04, about 0.05, about 0.06, about 0.07, about 0.08, about 0.09, about 0.1, about 0.2, about 0.3, about 0.4, about 0.5, about 0.6, about 0.7, about 0.8, about 0.9, about 1, about 1.1, about 1.2, about 1.3, about 1.4, about 1.5, about 1.6, about 1.7, about 1.8, about 1.9, about 2, about 2.1, about 2.2, about 2.3, about 2.4, about 2.5, about 2.6, about 2.7, about 2.8, about 2.9, about 3, from about 0 to about 3, from about 0 to about 2.5, from about 0 to about 2, from about 0 to about 1.5, from about 0 to about 1, from about 0 to about 0.5, from about 0 to about 0.3, from about 0 to about 0.2, from about 0 to about 0.1, from about 0 to about 0.05, from about 0 to about 0.01, from about 0 to about 0.001, no more than about 3, no more than about 2, no more than about 1.5, no more than about 1, no more than about 0.5, no more than about 0.3, no more than about 0.2, no more than about 0.1, no more than about 0.05, no more than about 0.01, or no more than about 0.001 gm total fatty acid/kg body weight of the animal.

Any suitable method can be used to determine or measure total fatty acid including but not limited to one or more of: extraction methods (e.g., solvent based extraction methods or nonsolvent liquid extraction methods such as the Babcock method or the Gerber method), hydrolysis methods, chemical techniques (e.g., Iodine value determination (e.g., using the Wijs method) or saponification number determination), mass spectrometry, gas chromatography, high pressure liquid chromatography, thin layer chromatography, nuclear magnetic resonance, or combinations thereof. In some embodiments, use of information provided by other sources such as but not limited to information found in books, in tables, using apps, using websites, or in packaging with a food item (such as nutritional labels including but not limited to those required by the US Food and Drug Administration, such as pursuant to 21 C.F.R. § 101 (e.g., § 101.9)) can be used as a method to determine total fatty acid. One or more of these methods can be used to determine or measure total fatty acid for one food item, for several food items, or for all the food items used to calculate the daily intake of total fatty acid.

The term “total monounsaturated fatty acid” as used herein is defined to include all cis monounsaturated fatty acids such as but not limited to oleic acid (18:1 n-9), myristoleic acid (14:1 n-7), palmitoleic acid (16:1 n-7), vaccenic acid (18:1 n-7), eicosenoic acid (20:1 n-9), erucic acid (22:1 n-9), nervonic acid (24:1 n-9), and those found in glycerides (e.g., triglycerides, diglycerides, and monoglycerides). Total monounsaturated fatty acid encompasses total oleic acid. In some embodiments, the method of treatment includes a daily intake of total monounsaturated fatty acid of no more than about 2 gm total monounsaturated fatty acid/kg body weight of the animal. In some embodiments, the method of treatment includes a daily intake of total monounsaturated fatty acid of about 0, about 0.001, about 0.01, about 0.02, about 0.03, about 0.04, about 0.05, about 0.06, about 0.07, about 0.08, about 0.09, about 0.1, about 0.2, about 0.3, about 0.4, about 0.5, about 0.6, about 0.7, about 0.8, about 0.9, about 1, about 1.1, about 1.2, about 1.3, about 1.4, about 1.5, about 1.6, about 1.7, about 1.8, about 1.9, about 2, from about 0 to about 2, from about 0 to about 1.5, from about 0 to about 1, from about 0 to about 0.5, from about 0 to about 0.3, from about 0 to about 0.2, from about 0 to about 0.1, from about 0 to about 0.05, from about 0 to about 0.01, from about 0 to about 0.001, no more than about 2, no more than about 1.5, no more than about 1, no more than about 0.5, no more than about 0.3, no more than about 0.2, no more than about 0.1, no more than about 0.05, no more than about 0.01, or no more than about 0.001 gm total monounsaturated fatty acid/kg body weight of the animal.

Any suitable method can be used to determine or measure total monounsaturated fatty acid including but not limited to one or more of: extraction methods (e.g., solvent based extraction methods or nonsolvent liquid extraction methods), hydrolysis methods, chemical techniques (e.g., Iodine value determination (e.g., using the Wijs method) or saponification number determination), mass spectrometry, gas chromatography, high pressure liquid chromatography, thin layer chromatography, nuclear magnetic resonance, or combinations thereof. In some embodiments, use of information provided by other sources such as but not limited to information found in books, in tables, using apps, using websites, or in packaging with a food item (such as nutritional labels including but not limited to those required by the US Food and Drug Administration, such as pursuant to 21 C.F.R. § 101 (e.g., § 101.9)) can be used as a method to determine total monounsaturated fatty acid. One or more of these methods can be used to determine or measure total monounsaturated fatty acid for one food item, for several food items, or for all the food items used to calculate the daily intake of total monounsaturated fatty acid.

The term “total oleic acid” as used herein is defined to include all forms of oleic acid ((9Z)-Octadec-9-enoic acid; CAS #112-80-1) and oleate (e.g., salts or esters), including those found in glycerides (e.g., triglycerides, diglycerides, and monoglycerides). In some embodiments, the method of treatment includes a daily intake of total oleic acid of no more than about 2 gm total oleic acid/kg body weight of the animal. In other embodiments, the method of treatment includes a daily intake of total oleic acid of about 0, about 0.001, about 0.01, about 0.02, about 0.03, about 0.04, about 0.05, about 0.06, about 0.07, about 0.08, about 0.09, about 0.1, about 0.2, about 0.3, about 0.4, about 0.5, about 0.6, about 0.7, about 0.8, about 0.9, about 1, about 1.1, about 1.2, about 1.3, about 1.4, about 1.5, about 1.6, about 1.7, about 1.8, about 1.9, about 2, from about 0 to about 2, from about 0 to about 1.5, from about 0 to about 1, from about 0 to about 0.5, from about 0 to about 0.3, from about 0 to about 0.2, from about 0 to about 0.1, from about 0 to about 0.05, from about 0 to about 0.01, from about 0 to about 0.001, no more than about 2, no more than about 1.5, no more than about 1, no more than about 0.5, no more than about 0.3, no more than about 0.2, no more than about 0.1, no more than about 0.05, no more than about 0.01, or no more than about 0.001 gm total oleic acid/kg body weight of the animal.

Any suitable method can be used to determine or measure total oleic acid including but not limited to one or more of: extraction methods (e.g., solvent based extraction methods or nonsolvent liquid extraction methods), hydrolysis methods, chemical techniques (e.g., Iodine value determination (e.g., using the Wijs method) or saponification number determination), mass spectrometry, gas chromatography, high pressure liquid chromatography, thin layer chromatography, nuclear magnetic resonance, or combinations thereof. In some embodiments, use of information provided by other sources such as but not limited to information found in books, in tables, using apps, using websites, or in packaging with a food item (such as nutritional labels including but not limited to those required by the US Food and Drug Administration, such as pursuant to 21 C.F.R. § 101 (e.g., § 101.9)) can be used as a method to determine total oleic acid. One or more of these methods can be used to determine or measure total oleic acid for one food item, for several food items, or for all the food items used to calculate the daily intake of total oleic acid.

Intake by the animal can occur using any suitable process or manner including but not limited to ingestion (e.g., eating or drinking), enteral feeding (e.g., a nasogastric tube, a nasoduodenal tube, a nasojejunal tube, a gastrostomy tube, a gastrojejunostomy tube, or a jejunostomy tube), parenteral nutritional feeding (e.g., total parenteral nutritional feeding), intradialytic parenteral nutritional feeding, or a combination thereof.

Daily intake of a substance is defined herein to mean the intake amount of that substance (e.g., in gm of substance per kg animal weight) over a twenty four hour period of time. The substance can be, for example, total fat, total fatty acid, total monounsaturated fatty acid, or total oleic acid.

Food is defined herein as any item processed, partially processed, or unprocessed for intake by an animal (e.g., human). Food includes but is not limited to any substance intended to be, or reasonably expected to be, ingested (e.g., by eating or by drinking, and also includes but is not limited to chewing gum, medicines, vitamins, or dietary or nutritional supplements) by an animal (e.g., a human), any substance for enteral feeding of an animal (e.g., a human), any substance for parenteral nutritional feeding of an animal (e.g., a human), or any substance for intradialytic parenteralal nutritional feeding of an animal (e.g., a human).

In some embodiments, the method of treating can include administering an SCD1 inhibitor to the animal (e.g., a mammal, rodent, primate, rat, mouse, or human) (as described herein) where the animal (as described herein) has a daily intake of one or more of (a) a specified amount of total fat (e.g., no more than about 3 gm/kg body weight of the animal), (b) a specified amount of total fatty acid (e.g., no more than about 3 gm/kg body weight of the animal), (c) a specified amount of total monounsaturated fatty acid (e.g., no more than about 2 gm/kg body weight of the animal), or (d) a specified amount of total oleic acid (e.g., no more than about 2 gm/kg body weight of the animal). In other embodiments, the animal is in need of the treatment. In certain instances, the SCD1 inhibitor is in a composition such as a pharmaceutical composition. In other instances, the amount of SCD1 inhibitor administered is an effective amount such as a therapeutically effective amount.

In some embodiments, the method of treating can include determining whether the SCD gene (e.g., the SCD1 gene) is absent from one or both chromatids in the animal (e.g., a mammal, rodent, primate, rat, mouse, or human), as described herein. If the SCD gene (e.g., the SCD1 gene) is absent from one or both chromatids, then the method further includes administering an SCD inhibitor (e.g., an SCD1 inhibitor) to the animal (e.g., a mammal, rodent, or primate) (as described herein) where the animal (as described herein) has a daily intake of one or more of (a) a specified amount of total fat (e.g., no more than about 3 gm/kg body weight of the animal), (b) a specified amount of total fatty acid (e.g., no more than about 3 gm/kg body weight of the animal), (c) a specified amount of total monounsaturated fatty acid (e.g., no more than about 2 gm/kg body weight of the animal), or (d) a specified amount of total oleic acid (e.g., no more than about 2 gm/kg body weight of the animal). In other embodiments, the animal is in need of the treatment. In certain instances, the SCD1 inhibitor is in a composition such as a pharmaceutical composition. In other instances, the amount of SCD1 inhibitor administered is an effective amount such as a therapeutically effective amount.

The presently-disclosed subject matter is further illustrated by the following specific but non-limiting examples. The following examples may include compilations of data that are representative of data gathered at various times during the course of development and experimentation related to the present invention.

### EXAMPLES

### SCD Expression

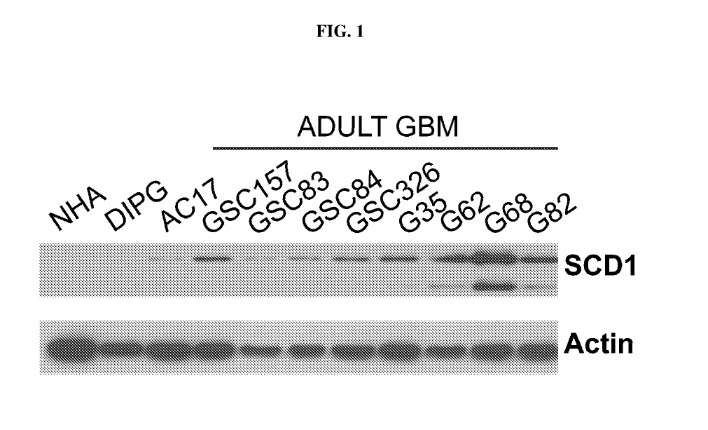

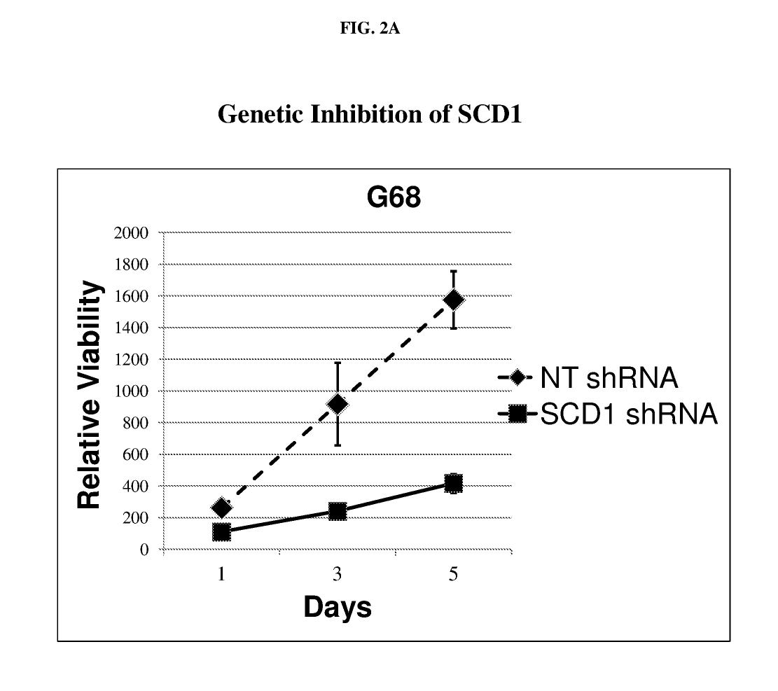

FIG. 1 shows SCD1 expression determined in primary gliobastoma multiforme (GBM) cells. Table 1 describes each cell line and its source.

TABLE 1

| | Description | |
|---|---|---|
| Cell Line | of Cell Line | Source of Cell Line |
| NHA | Normal Human | Commercial |
| | Astrocytes | |
| DIPG | Diffuse Intrinsic | Biplab Dasgupta Laboratory at |
| | Pontine Glioma - a | Cincinnati Children's Hospital |
| | pediatric primary | Medical Center |
| | GBM cell line | |
| AC17 | Primary Adult | Ichiro Nakano laboratory (formerly |
| | Glioblastoma | at the Ohio State University; now at |
| | multiforme | University of Alabama at |
| | | Birmingham) |
| GSC157 | Primary Adult | Ichiro Nakano laboratory (formerly |
| | Glioblastoma | at the Ohio State University; now at |
| | multiforme | University of Alabama at |
| | | Birmingham) |
| GSC83 | Primary Adult | Ichiro Nakano laboratory (formerly |
| | Glioblastoma | at the Ohio State University; now at |
| | multiforme | University of Alabama at |
| | | Birmingham) |
| GSC84 | Primary Adult | Ichiro Nakano laboratory (formerly |
| | Glioblastoma | at the Ohio State University; now at |
| | multiforme | University of Alabama at |
| | | Birmingham) |
| GSC326 | Primary Adult | Ichiro Nakano laboratory (formerly |
| | Glioblastoma | at the Ohio State University; now at |
| | multiforme | University of Alabama at |
| | | Birmingham) |
| GBM157 | Primary Adult | Ichiro Nakano laboratory (formerly |
| | Glioblastoma | at the Ohio State University; now at |
| | multiforme | University of Alabama at |
| | | Birmingham) |
| G35 | Primary Adult | Christoph Beier, RWTH Aachen, |
| | Glioblastoma | Medical School, Germany |
| | multiforme | |
| G62 | Primary Adult | Christoph Beier, RWTH Aachen, |
| | Glioblastoma | Medical School, Germany |
| | multiforme | |
| G68 | Primary Adult | Christoph Beier, RWTH Aachen, |
| | Glioblastoma | Medical School, Germany |
| | multiforme | |
| G82 | Primary Adult | Christoph Beier, RWTH Aachen, |
| | Glioblastoma | Medical School, Germany |
| | multiforme | |

In the top gel, the bands are two isoforms of SCD. In the second gel, the actin band is used as a protein loading control. These gels show expression of SCD in adult primary GBM lines, but weak or undetectable expression in normal human astrocytes and in a pediatric glioma primary cell line. Also, the expression of SCD including expression of its two isoforms is variable among adult GBM cell lines.

### SCD Inhibition

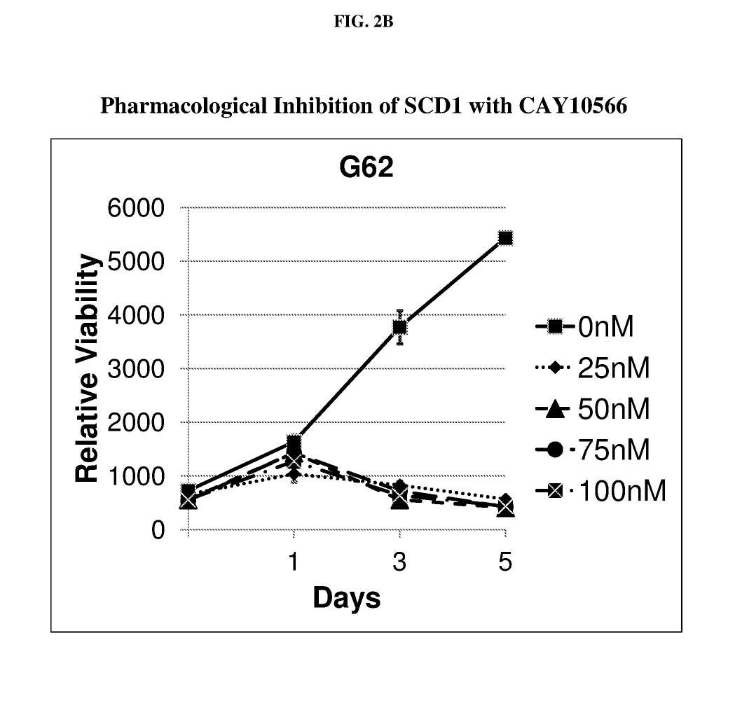

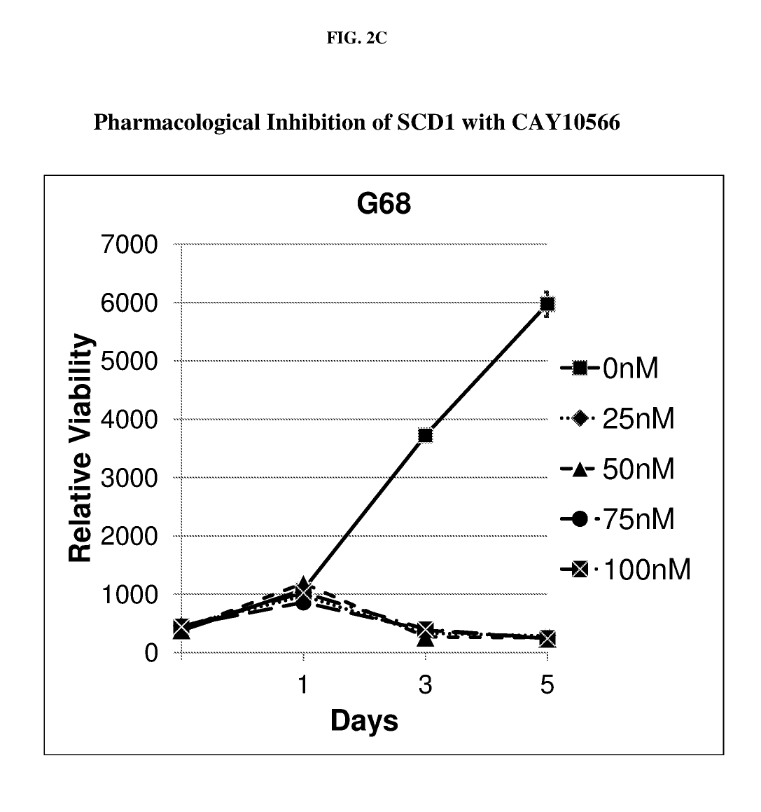

FIG. 2 shows the results of inhibition of SCD1 using gene silencing and using a small molecule inhibitor.

For the genetic inhibition studies of FIG. 2A, G68 cells were infected with control (NT=nontarget) shRNA or SCD1 shRNA using lentiviral transduction. Two days after viral transduction, an equal number of cells was seeded in 96 wells in quadruplicate. Viable cells were quantified at the indicated times using CellTiter Glo cell viability assay kit (Promega).

For the pharmacological inhibition studies of FIGS. 2B and 2C, an equal number of G62 cells was seeded in 96 wells in the presence of SCD1 inhibitor or vehicle (DMSO) in quadruplicate. Viable cells were quantified at indicated times using CellTiter Glo cell viability assay kit (Promega).

Cell viability assays in the presence or absence of SCD1 shRNA show that genetic inhibition of SCD1 resulted in induction of cell death and growth inhibition, as demonstrated in FIG. 2A. Cell viability assays in the presence or absence of CAY10566 (3-[4-(2-chloro-5-fluorophenoxy)-1-piperidinyl]-6-(5-methyl-1,3,4-oxadiazol-2-yl)-pyridazine; CAS Registry number of 944808-88-2) show that pharmacological inhibition of SCD1 resulted in induction of G62 cell death and growth inhibition, as demonstrated in FIG. 2B. Cell viability assays in the presence or absence of CAY10566 show that pharmacological inhibition of SCD1 resulted in induction of G68 cell death and growth inhibition, as demonstrated in FIG. 2C.

### Oleate Rescue of SCD-Inhibition

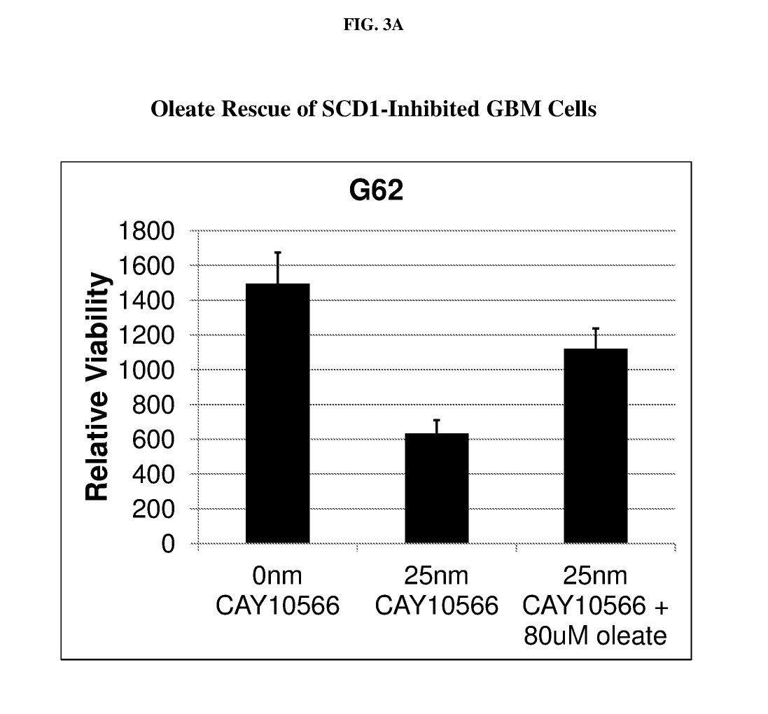

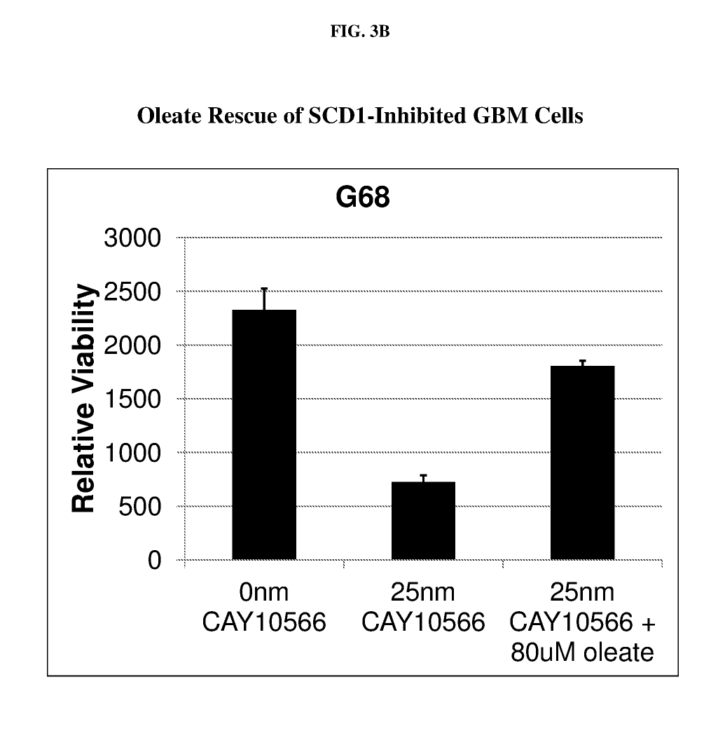

FIG. 3 shows SCD1-inhibited GBM cells were rescued by oleic acid conjugated to BSA (purchased from Sigma). Oleic acid is the product of the enzymatic reaction catalyzed by SCD1. Oleic acid is conjugated to BSA to solubilize the oleic acid; the BSA is cleaved from oleic acid upon entry into the cell. Equal number of cells was seeded in 96 wells in the presence of (each in quadruplicate) (a) vehicle (DMSO) (“0 nm CAY 10566”), (b) 25 nM of SCD1 inhibitor only (“25 nm CAY 10566”), or (c) 25 nM of SCD1 inhibitor plus 80 μM BSA-Oleate (“25 nm CAY 10566+80 uM oleate”). Viable cells were quantified after 72 hours using CellTiter Glo cell viability assay kit (Promega).

FIG. 3A shows that BSA-conjugated oleic acid rescued SCD1-inhibited G62 Cells. The G62 cell viability assays were performed in the presence or absence of CAY10566 and in the presence or absence of BSA-conjugated oleic acid. The increased viability of the cells treated with both CAY10566 and BSA-conjugated oleic acid compared to cells treated with only CAY10566 show that SCD1-inhibited G62 cells can be partly rescued by BSA-conjugated oleic acid.

FIG. 3B shows that BSA-conjugated oleic acid rescued SCD1-inhibited G68 Cells. The G68 cell viability assays were performed in the presence or absence of CAY10566 and in the presence or absence of BSA-conjugated oleic acid. The increased viability of the cells treated with both CAY10566 and BSA-conjugated oleic acid compared to cells treated with only CAY10566 show that SCD1-inhibited G68 cells can be partly rescued by BSA-conjugated oleic acid.

### Sensitivity of High SCD1 GBM Stem Cell Lines to SCD1 Inhibitor

For these viability experiments, 5000 cells were seeded in quadruplicate in serum-free DMEM-F/12 medium supplemented with B27 and two growth factors (EGF and bFGF). The cells were allowed to settle for 24 hours, then drug (or vehicle=DMSO) was added at indicated concentrations. The cell viability assay was performed after 72 hours using the CellTiter-Glow reagent (Promega).

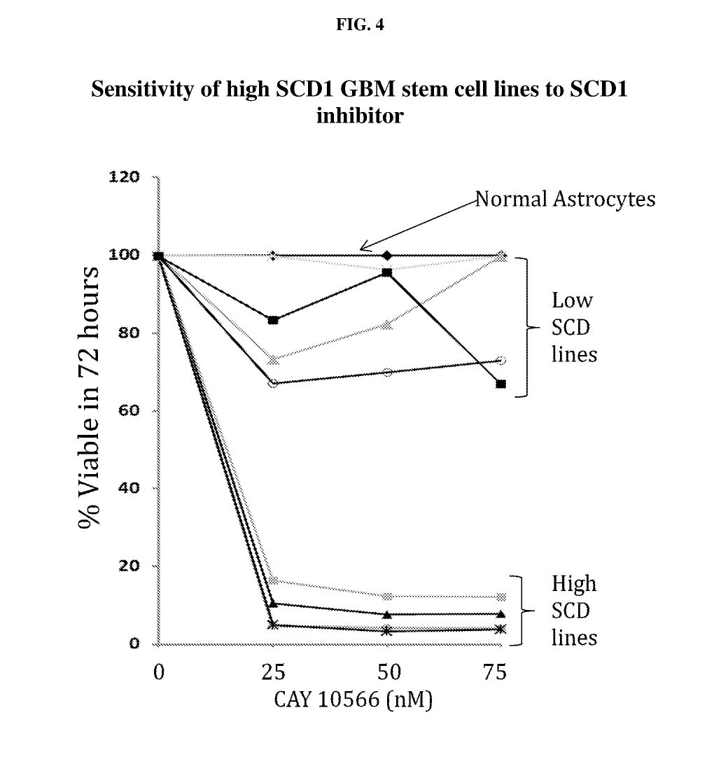

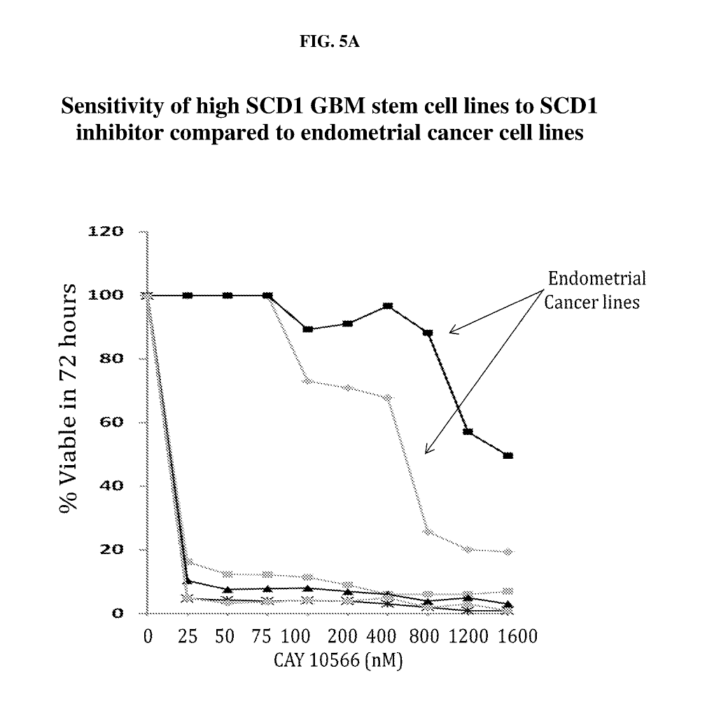

FIG. 4 shows inhibition of different cell lines when exposed to an SCD1 inhibitor at varying concentrations. High SCD cells express SCD from one copy of Chromosome 10 only due to co-deletion of the other copy of SCD with the PTEN deletion. Low SCD cells have one copy of SCD co-deleted with PTEN; expression from the other copy is absent (e.g., due to methylation). The different cells lines are: black diamond, normal human astrocytes (see Table 1); gray cross, GSC157 (see Table 1); black square, TS600 (Cameron Brennan, Memorial Sloan Kettering, N.Y.); gray triangle, GBM39 (Jann Sarkaria, Mayo Clinic, Rochester, Minn.); open circle, TS1156 (Cameron Brennan, Memorial Sloan Kettering, N.Y.); gray square, GBM157 (see Table 1); black triangle, G62 (see Table 1); gray circle, G68 (see Table 1); and black cross, G82 (see Table 1).

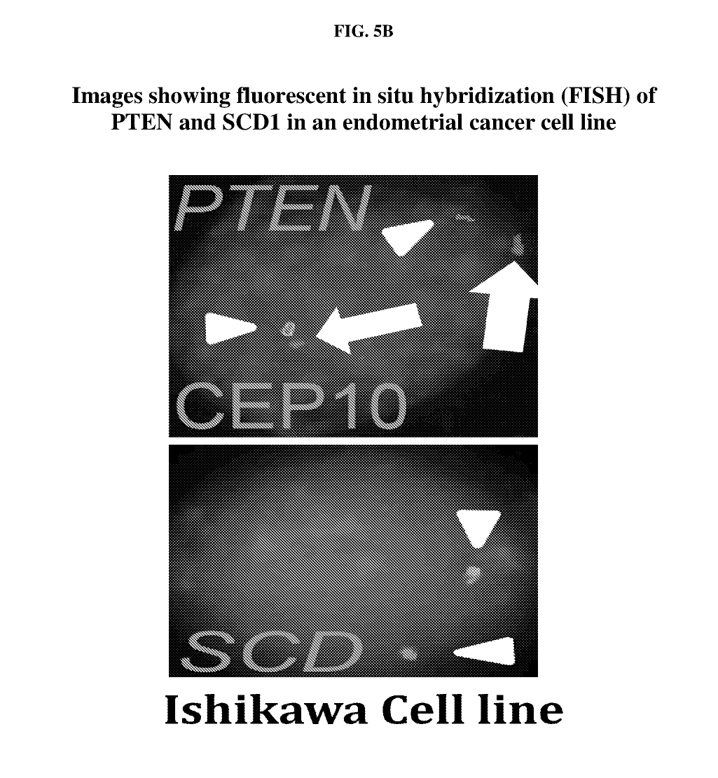

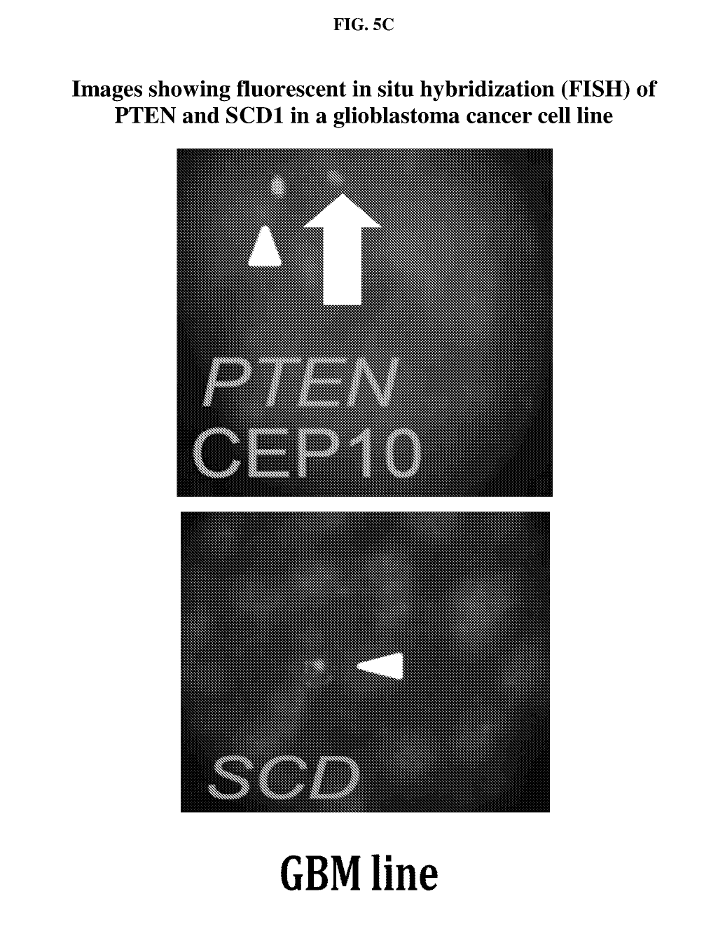

FIG. 5A shows sensitivity of GBM stem cell lines to SCD1 inhibitor compared to endometrial cancer cell lines. The cell viability experiments were performed as described above using the indicated SCD1 inhibitor concentrations and the indicated cell lines. The cells lines are: black square, Ishikawa cell line (from ATCC); gray diamond, HEC50B (from ATCC); gray square, GBM157 (see Table 1); black triangle, G62 (see Table 1); gray circle, G68 (see Table 1); and black cross, G82 (see Table 1). FIGS. 5B and 5C are images showing Fluorescent In Situ Hybridization (FISH) of the PTEN gene and the SCD1 gene in an endometrial cancer cell line (the Ishikawa cell line) and in a glioblastoma cancer cell line (the G62 cell line), respectively. Standard methods at the Genetics Core at Cincinnati Children's Hospital Medical Center were used to perform FISH with probes purchased from commercial sources. The arrowheads point to fluorescence resulting from hybridization with the PTEN gene or with the SCD1 gene, as indicated. The arrows point to the chromosome 10 centromere.

These data show that there is a loss of one copy of PTEN and SCD in the primary GBM line, whereas two copies of SCD and PTEN are present in the endometrial cancer cell line.

The headings used in the disclosure are not meant to suggest that all disclosure relating to the heading is found within the section that starts with that heading. Disclosure for any subject may be found throughout the specification.

It is noted that terms like “preferably,” “commonly,” and “typically” are not used herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

As used in the disclosure, “a” or “an” means one or more than one, unless otherwise specified. As used in the claims, when used in conjunction with the word “comprising” the words “a” or “an” means one or more than one, unless otherwise specified. As used in the disclosure or claims, “another” means at least a second or more, unless otherwise specified. As used in the disclosure, the phrases “such as”, “for example”, and “e.g.” mean “for example, but not limited to” in that the list following the term (“such as”, “for example”, or “e.g.”) provides some examples but the list is not necessarily a fully inclusive list. The word “comprising” means that the items following the word “comprising” may include additional unrecited elements or steps; that is, “comprising” does not exclude additional unrecited steps or elements.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

Detailed descriptions of one or more embodiments are provided herein. It is to be understood, however, that the present invention may be embodied in various forms. Therefore, specific details disclosed herein (even if designated as preferred or advantageous) are not to be interpreted as limiting, but rather are to be used as an illustrative basis for the claims and as a representative basis for teaching one skilled in the art to employ the present invention in any appropriate manner. Indeed, various modifications of the invention in addition to those described herein will become apparent to those skilled in the art from the foregoing description and the accompanying figures. Such modifications are intended to fall within the scope of the appended claims.

What is claimed is:

1. A method for treating cancer in an animal comprising
administering an SCD inhibitor to the animal,
wherein an SCD gene is absent from one or both chromatids.

2. The method of claim 1, wherein the SCD inhibitor is an SCD1 inhibitor.

3. The method of claim 1 or claim 2, wherein the SCD inhibitor is a small molecule SCD1 inhibitor.

4. The method of any of claims 1 to 3, wherein the SCD inhibitor is 3-[4-(2-chloro-5-fluorophenoxy)-1-piperidinyl]-6-(5-methyl-1,3,4-oxadiazol-2-yl)-pyridazine.

5. The method of any of claims 1 to 4, wherein the SCD gene is an SCD1 gene.

6. The method of any of claims 1 to 5, wherein the method further comprises
determining if the SCD gene is absent from one or both chromatids.

7. The method of any of claims 1 to 5, wherein prior to the administering step, the method further comprises
determining if the SCD gene is absent from one or both chromatids.

8. The method of claim 6 or claim 7, wherein the absence of the SCD gene from one or both chromatids is determined by measuring the presence or absence of the PTEN gene in one or both chromatids.

9. The method of any of claims 6 to 8, wherein the absence of the SCD gene from one or both chromatids is determined by measuring the presence or absence of the SCD gene in one or both chromatids.

10. The method of any of claims 6 to 9, wherein the measuring the presence or absence of the SCD gene, the presence or absence of the PTEN gene, or both are performed using FISH, CISH, or a CNV array.

11. The method of any of claims 1 to 10, wherein the animal has a daily intake of one or more of (a) total fat of no more than about 3 gm/kg body weight, (b) total fatty acid of no more than about 3 gm/kg body weight, (c) total monounsaturated fatty acid of no more than about 2 gm/kg body weight, or (d) total oleic acid of no more than about 2 gm/kg body fat.

12. The method of any of claims 1 to 11, wherein the animal has a daily intake of one or more of (a) total fat of no more than about 1 gm/kg body weight, (b) total fatty acid of no more than about 1 gm/kg body weight, (c) total monounsaturated fatty acid of no more than about 1 gm/kg body weight, or (d) total oleic acid of no more than about 1 gm/kg body fat.

13. The method of any of claims 1 to 12, wherein the animal has a daily intake of one or more of (a) total fat of no more than about 0.1 gm/kg body weight, (b) total fatty acid of no more than about 0.1 gm/kg body weight, (c) total monounsaturated fatty acid of no more than about 0.1 gm/kg body weight, or (d) total oleic acid of no more than about 0.1 gm/kg body fat.

14. The method of any of claims 1 to 13, wherein the animal has a daily intake of one or more of (a) total fat of no more than about 0.01 gm/kg body weight, (b) total fatty acid of no more than about 0.01 gm/kg body weight, (c) total monounsaturated fatty acid of no more than about 0.01 gm/kg body weight, or (d) total oleic acid of no more than about 0.01 gm/kg body fat.

15. The method of any of claims 11 to 14, wherein the intake occurs by ingestion, enteral feeding, parenteral feeding, or a combination thereof.

16. The method of any of claims 1 to 15, wherein the animal is human and the one or both chromatids are part of chromosome 10.

17. The method of any of claims 1 to 16, wherein the animal is in need of the treatment.

18. The method of any of claims 1 to 17, wherein the method is for treating glioblastoma multiforme, endometrial cancer, melanoma, prostate cancer, lung cancer, breast cancer, kidney cancer, or bladder cancer.

19. The method of any of claims 1 to 18, wherein the method is for treating glioblastoma multiforme, endometrial cancer, or melanoma.

20. The method of any of claims 1 to 19, wherein the method is for treating cancerous tumors, cancers that are inherited, or cancers resulting from an inherited predisposition.

21. The method of any of claims 1 to 20, wherein the method is for treating brain tumors.

22. The method of any of claims 1 to 21, wherein the animal is a mammal, rodent, primate, rat, mouse, or human.

23. The method of any of claims 1 to 22, wherein the SCD inhibitor is administered to the animal in an amount of from about 10 mg of SCD inhibitor/kg animal body weight to about 200 mg of SCD inhibitor/kg animal body weight.

24. The method of any of claims 1 to 23, wherein the administering comprises parenteral administration, a mucosal administration, intravenous administration, subcutaneous administration, topical administration, intradermal administration, oral administration, sublingual administration, intranasal administration, or intramuscular administration.

25. The method of any of claims 1 to 24, wherein the treatments further comprises one or more of surgical intervention, chemotherapy, radiation therapy, hormone therapies, immunotherapy, or adjuvant systematic therapies.

26. A method for treating cancer in an animal comprising
administering an SCD inhibitor to the animal;
wherein the animal has a daily intake of one or more of (a) total fat of no more than about 3 gm/kg body weight, (b) total fatty acid of no more than about 3 gm/kg body weight, (c) total monounsaturated fatty acid of no more than about 2 gm/kg body weight, or (d) total oleic acid of no more than about 2 gm/kg body fat.

27. The method of claim 26, wherein the method further comprises
determining if the SCD gene is absent from one or both chromatids.

28. The method of claim 26, wherein prior to the administering step, the method further comprises
determining if the SCD gene is absent from one or both chromatids.

29. The method of claim 27 or claim 28, wherein the absence of the SCD gene from one or both chromatids is determined by measuring the presence or absence of the PTEN gene in one or both chromatids.

30. The method of any of claims 27 to 29, wherein the absence of the SCD gene from one or both chromatids is determined by measuring the presence or absence of the SCD gene in one or both chromatids.

31. The method of any of claims 27 to 30, wherein the measuring the presence or absence of the SCD gene, the presence or absence of the PTEN gene, or both are performed using FISH, CISH, or a CNV array.

32. The method of any of claims 26 to 31, wherein the SCD gene is absent from one or both chromatids.

33. The method of any of claims 26 to 32, wherein the animal has a daily intake of one or more of (a) total fat of no more than about 1 gm/kg body weight, (b) total fatty acid of no more than about 1 gm/kg body weight, (c) total monounsaturated fatty acid of no more than about 1 gm/kg body weight, or (d) total oleic acid of no more than about 1 gm/kg body fat.

34. The method of any of claims 26 to 33, wherein the animal has a daily intake of one or more of (a) total fat of no more than about 0.1 gm/kg body weight, (b) total fatty acid of no more than about 0.1 gm/kg body weight, (c) total monounsaturated fatty acid of no more than about 0.1 gm/kg body weight, or (d) total oleic acid of no more than about 0.1 gm/kg body fat.

35. The method of any of claims 26 to 34, wherein the animal has a daily intake of one or more of (a) total fat of no more than about 0.01 gm/kg body weight, (b) total fatty acid of no more than about 0.01 gm/kg body weight, (c) total monounsaturated fatty acid of no more than about 0.01 gm/kg body weight, or (d) total oleic acid of no more than about 0.01 gm/kg body fat.

36. The method of any of claims 26 to 35, wherein the intake occurs by ingestion, enteral feeding, parenteral feeding, or a combination thereof.

37. The method of any of claims 26 to 36, wherein the animal is human and the one or both chromatids are part of chromosome 10.

38. The method of any of claims 26 to 37, wherein the animal is in need of the treatment.

39. The method of any of claims 26 to 38, wherein the method is for treating glioblastoma multiforme, endometrial cancer, melanoma, prostate cancer, lung cancer, breast cancer, kidney cancer, or bladder cancer.

40. The method of any of claims 26 to 39, wherein the method is for treating glioblastoma multiforme, endometrial cancer, or melanoma.

41. The method of any of claims 26 to 40, wherein the method is for treating cancerous tumors, cancers that are inherited, or cancers resulting from an inherited predisposition.

42. The method of any of claims 26 to 41, wherein the method is for treating brain tumors.

43. The method of any of claims 27 to 42, wherein the SCD gene is an SCD1 gene.

44. The method of any of claims 26 to 43, wherein the animal is a mammal, rodent, primate, rat, mouse, or human.

45. The method of any of claims 26 to 44, wherein the SCD inhibitor is a small molecule SCD1 inhibitor.

46. The method of any of claims 26 to 45, wherein the SCD inhibitor is an SCD1 inhibitor.

47. The method of any of claims 26 to 46, wherein the SCD inhibitor is 3-[4-(2-chloro-5-fluorophenoxy)-1-piperidinyl]-6-(5-methyl-1,3,4-oxadiazol-2-yl)-pyridazine.

48. The method of any of claims 26 to 47, wherein the SCD inhibitor is administered to the animal in an amount of from about 10 mg of SCD inhibitor/kg animal body weight to about 200 mg of SCD inhibitor/kg animal body weight.

49. The method of any of claims 26 to 48, wherein the administering comprises parenteral administration, a mucosal administration, intravenous administration, subcutaneous administration, topical administration, intradermal administration, oral administration, sublingual administration, intranasal administration, or intramuscular administration.

50. The method of any of claims 26 to 49, wherein the treatments further comprises one or more of surgical intervention, chemotherapy, radiation therapy, hormone therapies, immunotherapy, or adjuvant systematic therapies.

51. A method for treating cancer in an animal comprising
determining whether the SCD gene is absent from one or both chromatids; and
administering an SCD inhibitor to the animal, where the animal has a daily intake of one or more of (a) total fat of no more than about 3 gm/kg body weight, (b) total fatty acid of no more than about 3 gm/kg body weight, (c) total monounsaturated fatty acid of no more than about 3 gm/kg body weight, or (d) total oleic acid of no more than about 2 gm/kg body fat;
wherein the step of administering is performed only if the SCD gene is absent from one or both chromatids, using the determining step.

52. The method of claim 51, wherein the absence of the SCD gene from one or both chromatids is determined by measuring the presence or absence of the PTEN gene in one or both chromatids.

53. The method of claim 51 or claim 52, wherein the absence of the SCD gene from one or both chromatids is determined by measuring the presence or absence of the SCD gene in one or both chromatids.

54. The method of any of claims 51 to 53, wherein the measuring the presence or absence of the SCD gene, the presence or absence of the PTEN gene, or both are performed using FISH, CISH, or a CNV array.

55. The method of any of claims 51 to 54, wherein the SCD gene is absent from one or both chromatids.

56. The method of any of claims 51 to 55, wherein the animal has a daily intake of one or more of (a) total fat of no more than about 1 gm/kg body weight, (b) total fatty acid of no more than about 1 gm/kg body weight, (c) total monounsaturated fatty acid of no more than about 1 gm/kg body weight, or (d) total oleic acid of no more than about 1 gm/kg body fat.

57. The method of any of claims 51 to 56, wherein the animal has a daily intake of one or more of (a) total fat of no more than about 0.1 gm/kg body weight, (b) total fatty acid of no more than about 0.1 gm/kg body weight, (c) total monounsaturated fatty acid of no more than about 0.1 gm/kg body weight, or (d) total oleic acid of no more than about 0.1 gm/kg body fat.

58. The method of any of claims 51 to 57, wherein the animal has a daily intake of one or more of (a) total fat of no more than about 0.01 gm/kg body weight, (b) total fatty acid of no more than about 0.01 gm/kg body weight, (c) total monounsaturated fatty acid of no more than about 0.01 gm/kg body weight, or (d) total oleic acid of no more than about 0.01 gm/kg body fat.

59. The method of any of claims 51 to 58, wherein the intake occurs by ingestion, enteral feeding, parenteral feeding, or a combination thereof.

60. The method of any of claims 51 to 59, wherein the animal is human and the one or both chromatids are part of chromosome 10.

61. The method of any of claims 51 to 60, wherein the animal is in need of the treatment.

62. The method of any of claims 51 to 61, wherein the method is for treating glioblastoma multiforme, endometrial cancer, melanoma, prostate cancer, lung cancer, breast cancer, kidney cancer, or bladder cancer.

63. The method of any of claims 51 to 62, wherein the method is for treating glioblastoma multiforme, endometrial cancer, or melanoma.

64. The method of any of claims 51 to 63, wherein the method is for treating cancerous tumors, cancers that are inherited, or cancers resulting from an inherited predisposition.

65. The method of any of claims 51 to 64, wherein the method is for treating brain tumors.

66. The method of any of claims 51 to 65, wherein the SCD gene is an SCD1 gene.

67. The method of any of claims 51 to 66, wherein the animal is a mammal, rodent, primate, rat, mouse, or human.

68. The method of any of claims 51 to 67, wherein the SCD inhibitor is an SCD1 inhibitor.

69. The method of any of claims 51 to 68, wherein the SCD inhibitor is a small molecule SCD1 inhibitor.

70. The method of any of claims 51 to 69, wherein the SCD inhibitor is 3-[4-(2-chloro-5-fluorophenoxy)-1-piperidinyl]-6-(5-methyl-1,3,4-oxadiazol-2-yl)-pyridazine.

71. The method of any of claims 51 to 70, wherein the SCD inhibitor is administered to the animal in an amount of from about 10 mg of SCD inhibitor/kg animal body weight to about 200 mg of SCD inhibitor/kg animal body weight.

72. The method of any of claims 51 to 71, wherein the administering comprises parenteral administration, a mucosal administration, intravenous administration, subcutaneous administration, topical administration, intradermal administration, oral administration, sublingual administration, intranasal administration, or intramuscular administration.

73. The method of any of claims 51 to 72, wherein the treatments further comprises one or more of surgical intervention, chemotherapy, radiation therapy, hormone therapies, immunotherapy, or adjuvant systematic therapies.
